(19) United States
(12) Patent Application Publication
(10) Pub. No.: US 2025/0000405 A1
(43) Pub. Date: Jan. 2, 2025

(54) PORTED CATHETERS CONFIGURED TO FACILITATE INSERTION OF BLOOD DRAW DEVICE

(71) Applicant: Becton, Dickinson and Company, Franklin Lakes, NJ (US)

(72) Inventors: Prasad Govindaraj, Tamil Nadu (IN); Meenakshi Sankar Gomathinayagam, Tamil Nadu (IN); Sridhaar Nandakumar, Tamil Nadu (IN); Jonathan Karl Burkholz, Salt Lake City, UT (US); Weston F. Harding, Lehi, UT (US); S. Ray Isaacson, Layton, UT (US); Curtis H. Blanchard, Riverton, UT (US)

(21) Appl. No.: 18/215,610

(22) Filed: Jun. 28, 2023

(51) Int. Cl. A61B 5/15

(52) U.S. Cl. CPC A61B 5/150992; A61M 25/0097; A61M 2039/0202; A61M 2039/0258; A61M 2039/0279; A61M 2039/0282

(57) ABSTRACT

Ported catheters can be configured to facilitate insertion of a blood draw device or other vascular probe or instrument. A ported catheter may include a bushing, or wedge, that is shaped to provide a smooth entry and advancement of a tube of a blood draw device that is coupled to the ported catheter, whether via a proximal opening or a port of the ported catheter. Such a bushing may be particularly beneficial when the port of the ported catheter is oriented at an angle towards the catheter. By incorporating such a bushing, a ported catheter may be better able to minimize the occurrence of kinking or bending of the tube of the blood draw device. A ported catheter may also include a catheter strain relief feature to minimize the occurrence of kinking or bending of the tube of the blood draw device while it is extended through the catheter.

### BACKGROUND

A ported catheter is a peripheral intravenous catheter (PIVC) having a port that is formed into the catheter adapter. FIG. 1 provides an example of a ported catheter 10. Ported catheter 10 includes a catheter adapter 11 having a proximal opening 12 and a support platform 15. A catheter 13 extends distally from catheter adapter 11. A port 14 extends upwardly from catheter adapter 11 and provides an access point, separate from proximal opening 12, into catheter 13. In FIG. 1, port 14 is oriented at a right angle relative to the longitudinal axis of catheter adapter 11. In other cases, a port could be oriented at an angle towards catheter 13. Although not shown, a ported catheter, such as ported catheter 10, may typically be initially integrated with a needle assembly for inserting the catheter into the patient's vasculature.

FIG. 2A provides an example of a blood draw device 20 that could be used with a PIVC including with a ported catheter. Blood draw device 20 includes a main body 21 having a distal tip 22 that is configured to insert into a catheter adapter of a PIVC (e.g., via a port, adapter or other opening) and a securing mechanism 25 that can couple main body 21 to the catheter adapter. A tube 23 extends through main body 21 and may be selectively advanced out through distal tip 22 via a slider 26. An adapter 24 can be connected to a proximal end of tube 23 and provides a means for connecting a blood draw set (e.g., a vacuum tube adapter) to tube 23.

FIG. 2B provides an example of how blood draw device 20 can be used with a PIVC 30. Distal tip 22 can be inserted into a proximal opening 32 of PIVC 30 until securing mechanism 25 is secured around proximal opening 32. Then, slider 26 can be advanced distally to cause tube 23 to pass through catheter adapter 31 and out through catheter 33 so that tube 23 can be positioned at a point within the vasculature where blood can be more effectively drawn. Because tube 23 is flexible, it can be difficult to advance tube 23 through catheter adapter 31 and into catheter 33. Once tube 23 is positioned appropriately, a syringe 40 or other mechanism can be used to draw blood through tube 23.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to ported catheters and more particularly to ported catheters that are configured to facilitate insertion of a blood draw device or other vascular probe or instrument. A ported catheter may include a bushing, or wedge, that is shaped to provide a smooth entry and advancement of a tube of a blood draw device that is coupled to the ported catheter, whether via a proximal opening or a port of the ported catheter. Such a bushing may be particularly beneficial when the port of the ported catheter is oriented at an angle towards the catheter. By incorporating such a bushing, a ported catheter may be better able to minimize the occurrence of kinking or bending of the tube of the blood draw device. A ported catheter may also include a catheter strain relief feature to minimize the occurrence of kinking or bending of the tube of the blood draw device while it is extended through the catheter.

In some embodiments of the present disclosure, a ported catheter may include a catheter adapter having a proximal opening, a catheter extending distally from the catheter adapter, a port, and a bushing having an inside surface that is configured to facilitate insertion of a tube of a blood draw device or other vascular probe or instrument into the catheter.

In some embodiments, the inside surface of the bushing may include a curved section.

In some embodiments, the curved section may have a concave cross-sectional shape.

In some embodiments, the inside surface of the bushing may include a proximal section positioned at a proximal opening of the bushing. The curved section may extend distally from the proximal section.

In some embodiments, the inside surface of the bushing may include a sloped section that extends proximally from the curved section.

In some embodiments, the port may be angled towards the bushing.

In some embodiments, the inside surface of the bushing may include a sloped section that extends between a distal end of the bushing and a proximal opening of the bushing.

In some embodiments, the sloped section may intersect with an outer surface of the bushing at the proximal opening.

In some embodiments, the inside surface of the bushing may include a curved section that extends between a distal end of the bushing and a proximal opening of the bushing. The curved section may have a convex cross-sectional shape.

In some embodiments, the inside surface may include a proximal section positioned at a proximal opening of the bushing and a sloped section that extends between the proximal section and a distal end of the bushing.

In some embodiments, a proximal end of the proximal section may be curved.

In some embodiments, the inside surface may include a proximal section positioned at a proximal opening of the bushing, a rounded section within the proximal section, a curved section that extends distally from the proximal section, and a sloped section that extends between the curved section and a distal end of the bushing.

In some embodiments, the bushing may be formed of plastic, metal, or rubber.

In some embodiments, the ported catheter may include a catheter strain relief feature formed around the catheter distal to the catheter adapter.

In some embodiments of the present disclosure, a bushing for a ported catheter may include a distal end that is configured to secure a catheter within a catheter adapter of the ported catheter, a proximal opening, and an inside surface that is configured to facilitate insertion of a tube of a blood draw device or other vascular probe or instrument into the catheter.

In some embodiments, the inside surface may include a curved section.

In some embodiments, the inside surface may include a sloped section and a proximal section. The curved section may be positioned between the sloped section and the proximal section.

In some embodiments, the curved section may extend between the proximal opening and the distal end.

In some embodiments, the inside surface may include a slanted section.

In some embodiments of the present disclosure, a system for collecting a blood sample may include a ported catheter and a blood draw device having a tube that is configured to extend through the ported catheter. The ported catheter may include a bushing having an inside surface that is configured to facilitate inserting the tube through the ported catheter.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 illustrates a prior art ported catheter.

FIG. 2A illustrates a prior art blood draw device.

FIG. 2B illustrates how the prior art blood draw device can be used with a PIVC to draw blood.

FIGS. 3A and 3B are cross-sectional views of a ported catheter that includes a bushing that is configured to facilitate insertion of a blood draw device through the ported catheter in accordance with one or more embodiments of the present disclosure.

FIG. 4A is a cross-sectional view of a ported catheter that includes a bushing that is configured to facilitate insertion of a blood draw device through the ported catheter in accordance with one or more embodiments of the present disclosure.

FIG. 4B is a cross-sectional view of a ported catheter that includes a bushing that is configured to facilitate insertion of a blood draw device through the ported catheter in accordance with one or more embodiments of the present disclosure.

FIG. 4C is a cross-sectional view of a ported catheter that includes a bushing that is configured to facilitate insertion of a blood draw device through the ported catheter in accordance with one or more embodiments of the present disclosure.

FIG. 4D is a cross-sectional view of a ported catheter that includes a bushing that is configured to facilitate insertion of a blood draw device through the ported catheter in accordance with one or more embodiments of the present disclosure.

FIG. 4E is a cross-sectional view of a ported catheter that includes a bushing that is configured to facilitate insertion of a blood draw device through the ported catheter in accordance with one or more embodiments of the present disclosure.

FIG. 4F is a cross-sectional view of a ported catheter that includes a bushing that is configured to facilitate insertion of a blood draw device through the ported catheter in accordance with one or more embodiments of the present disclosure.

FIG. 4G is a cross-sectional view of a ported catheter that includes a bushing that is configured to facilitate insertion of a blood draw device through the ported catheter in accordance with one or more embodiments of the present disclosure.

FIGS. 5A and 5B are side and cross-sectional views respectively of a bushing that is configured to facilitate insertion of a blood draw device through a ported catheter in which the bushing may be placed in accordance with one or more embodiments of the present disclosure.

FIG. 6 is a cross-sectional view of a ported catheter that includes a bushing that is configured to facilitate insertion of a blood draw device through the ported catheter and that includes a catheter strain relief feature in accordance with one or more embodiments of the present disclosure.

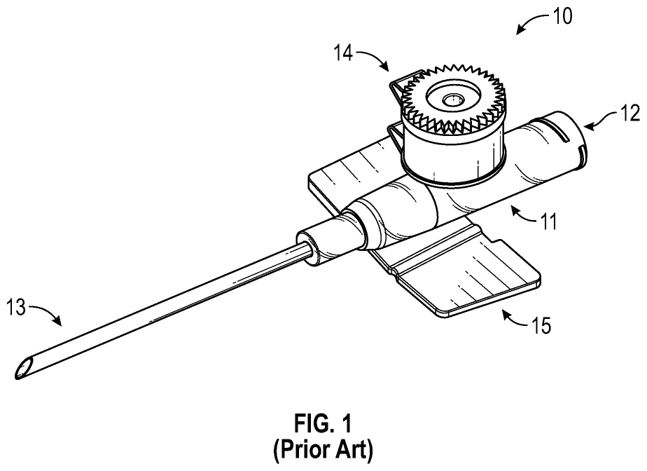

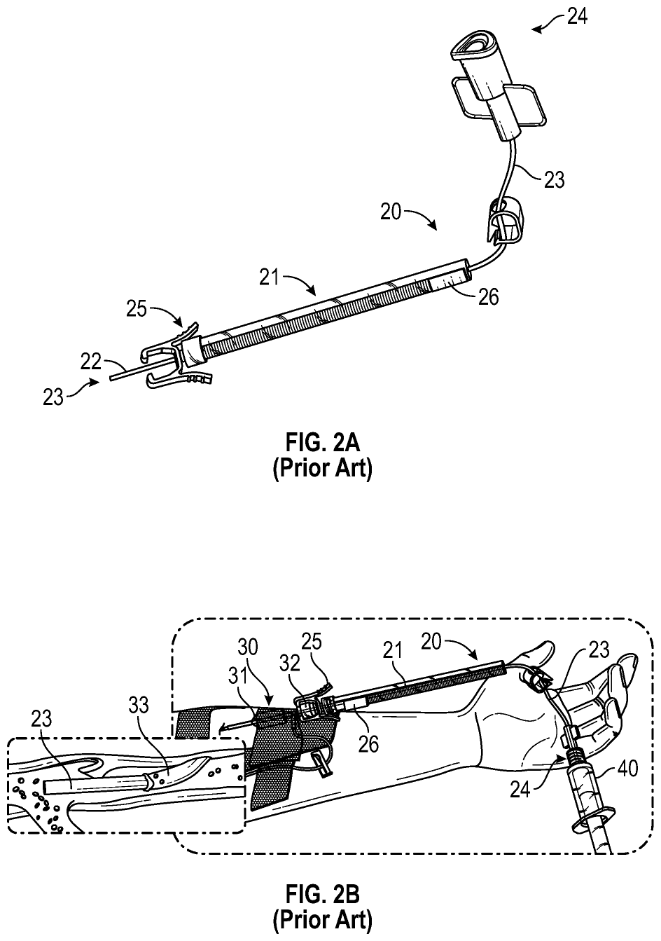

### DESCRIPTION OF EMBODIMENTS

In the specification and the claims, the term “bushing” should be construed as a component of a ported catheter that is separate from the catheter adapter and is positioned within a distal end of the catheter adapter to retain the catheter within the catheter adapter. For example, a distal end of the bushing may be configured to insert into a proximal end of the catheter and may cause the proximal end of the catheter to be pinched between the bushing and the catheter adapter when the bushing is fully inserted into the catheter adapter. Accordingly, a bushing may include a proximal opening, a distal opening and a lumen that extends therebetween to provide a pathway from the lumen of the catheter adapter into the catheter. Although embodiments of the present disclosure are described primarily in the context of a blood draw device, such embodiments can also facilitate insertion of another vascular probe or instrument (e.g., sensing probes, guidewires, etc.).

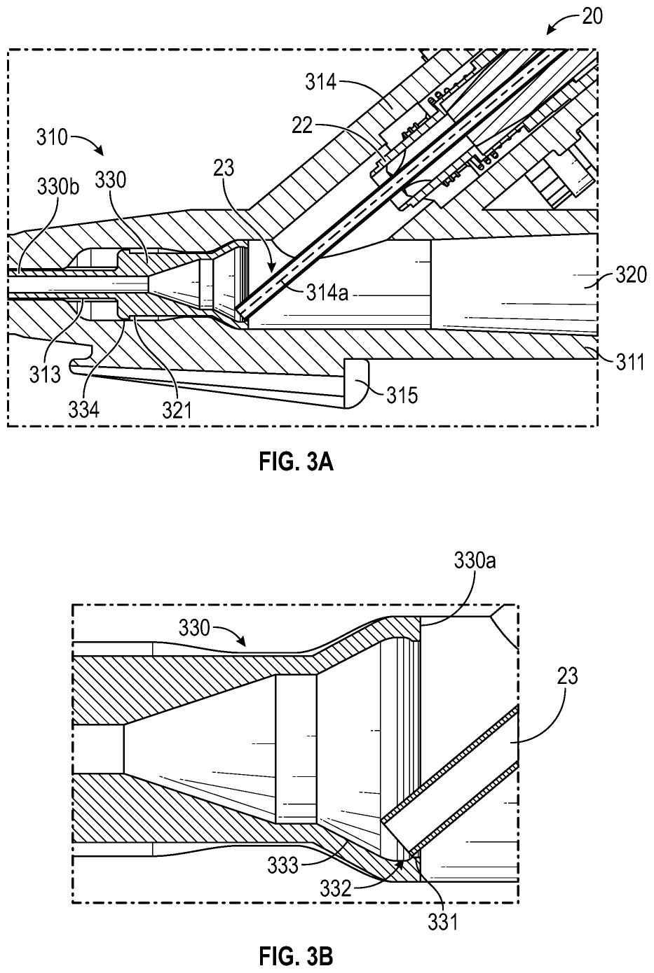

FIGS. 3A and 3B are cross-sectional views of a ported catheter 310 that includes a bushing 330 that is configured to facilitate insertion of a blood draw device 20 through ported catheter 310 in accordance with one or more embodiments of the present disclosure. Ported catheter 310 includes a catheter adapter 311, a proximal opening (not visible), a catheter 313, a port 314, and a stabilization platform 315.

Bushing 330 includes a distal end 330b that is inserted into the proximal end of catheter 313 and functions to retain catheter 313 within lumen 320 of catheter adapter 311. Bushing 330 can also be configured to remain secured within lumen 320 of catheter adapter 311. For example, in the depicted embodiment, bushing 330 includes a proximal-facing ridge 334 that contacts a distally-facing ridge 321 formed around lumen 320 of catheter adapter 311 to prevent bushing 330 from moving proximally within lumen 320. Proximal-facing ridge 334 can be positioned on bushing 330 so that the widened outer surface of bushing 330 at and towards proximal opening 330a abuts the widened section of lumen 320. In other embodiments, bushing 330, or any other bushing described herein, could be configured to form a friction fit with catheter adapter 311, could be adhered or welded within catheter adapter 311, or could otherwise be secured within catheter adapter 311.

Port 314 is oriented at an angle that is towards the distal end of catheter adapter 311. Accordingly, with blood draw device 20 coupled to port 314 and tube 23 advanced out from distal tip 22, tube 23 will extend towards bushing 330 along an axis 314a that intersects with proximal opening 330a. In other words, as tube 23 is advanced distally, tube 23 will contact an inside surface of bushing 330.

As is best shown in FIG. 3B, the inside surface of bushing 330 at proximal opening 330a can include a proximal section 331 that may form a lip around proximal opening 330a, a curved section 332 that may form a recessed ring around and distal to proximal section 331 (e.g., proximal section 331 can have a smaller diameter than curved section 332 and curved section 332 can have a convex cross-sectional shape), and a sloped section 333 that forms a distally decreasing diameter region proximal to curved section 332. Accordingly, as tube 23 is advanced distally within catheter adapter 311, the distal tip of tube 23 may initially contact proximal section 331 or possibly curved section 332. Then, as tube 23 is further advanced, the curved cross-sectional shape of curved section 332 can facilitate advancing the tip of tube 23 along a curved path to reach sloped section 333 (e.g., to facilitate bending/curving the tip of tube 23 upwardly relative to the orientation represented in FIG. 3B). Then, sloped section 333 can facilitate advancing the tip of tube 23 distally towards the narrowed portion of the lumen of bushing 330 that passes through distal end 330b and into catheter 313.

Curved section 332 can minimize the likelihood that the distal end of tube 23 will kink as it is advanced within catheter adapter 311, including in the depicted embodiments where port 314 is angled and in embodiments where port 314 may be oriented perpendicular to the longitudinal access of catheter adapter 311. For example, tube 23 is most likely to kink due to the bending that must occur to reach catheter 313 and this bending necessarily must occur as tube 23 contacts the inside surface of bushing 330. Curved section 332 can guide this bending along a curved path as the tip of tube 23 initially advances distally while contacting the inside surface of bushing 330. Curved section 332 also provides a less abrupt transition to sloped section 333.

Bushing 330 can also facilitate inserting tube 23 when blood draw device 20 is coupled to the proximal opening of ported catheter 310. In such cases, tube 23 can be advanced until contacting sloped section 333. Sloped section 333 can then facilitate advancing the tip of tube 23 towards distal end 330b and into catheter 313.

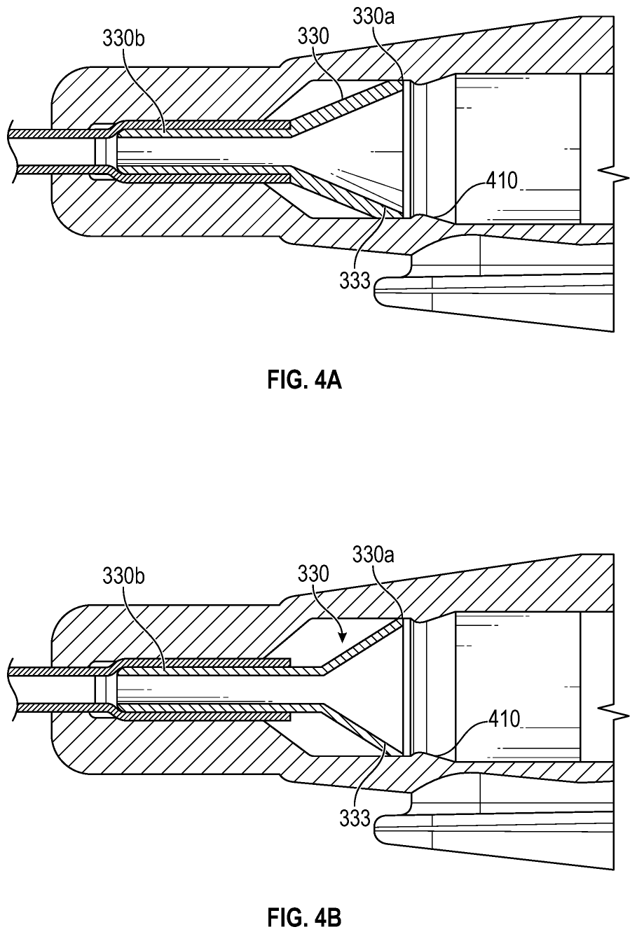

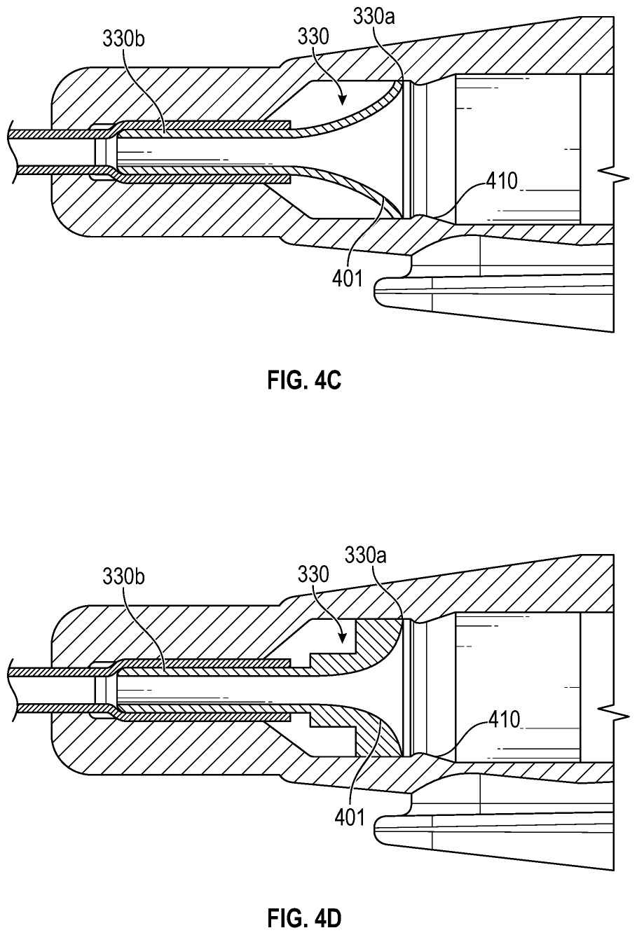

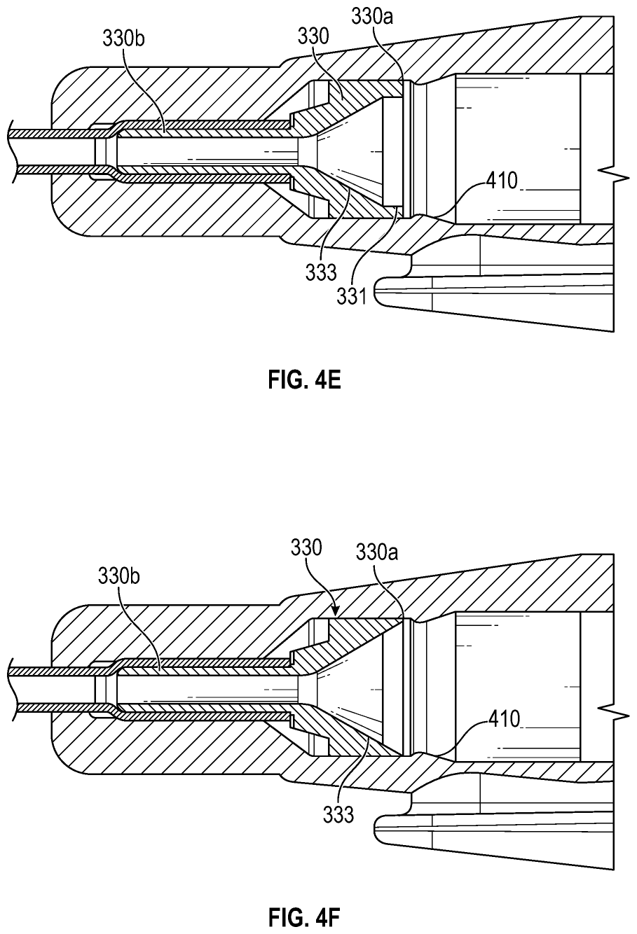

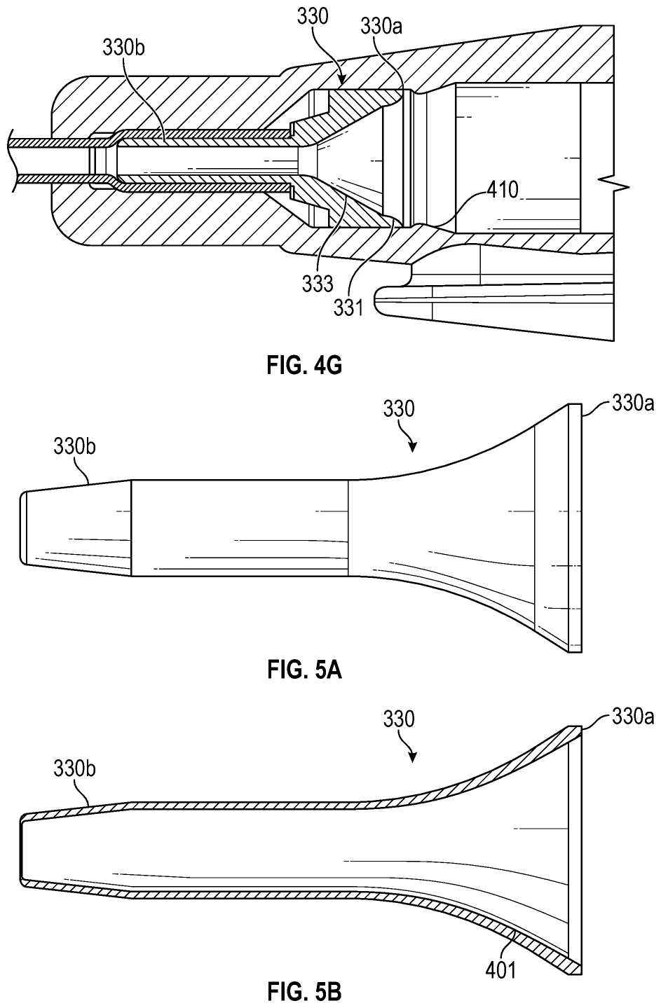

FIGS. 4A-4G each provide an example of a variation of bushing 330 that could be used in a ported catheter, or another PIVC, to facilitate insertion of a blood draw device through the ported catheter, or other PIVC, in accordance with embodiments of the present disclosure. The embodiments of bushing 330 represented in FIGS. 4A-4G could be used to facilitate insertion of tube 23 when blood draw device 20 is connected to a port of a ported catheter but may be particularly beneficial when blood draw device 20 is connected to the proximal opening of the ported catheter or other PIVC. Proximal opening 330a of bushing 330 can be positioned against a protrusion 410 in the lumen of the catheter adapter. Protrusion 410 can have a sloped proximal face that can facilitate directing tube 23 into proximal opening 330a.

In FIGS. 4A and 4B, bushing 330 includes a sloped section 333 that extends between distal end 330b and proximal opening 330a. In FIG. 4A, sloped section 333 is longer (i.e., the angle of sloped section 333 relative to the longitudinal axis of the catheter adapter is smaller) in comparison to sloped section in FIG. 4B. For example, in FIG. 4A, bushing 330 can be configured so that distal end 330b fully inserts into the narrowed section of the lumen of the catheter adapter, whereas, in FIG. 4B, bushing 330 can be configured so that distal end 330b extends proximally out from this narrowed section.

In FIGS. 4C and 4D, bushing 330 includes a curved section 401 that extends between distal end 330b and proximal opening 330a. Curved section 401 can have a concave cross-sectional shape such that the inside diameter of bushing decreases along a curve from proximal opening 330a towards distal end 330b. The design of bushing 330 in FIG. 4D includes thickened walls and may be suitable when bushing 330 is formed of a rubberized material. For example, the thickness of the walls can be configured to control the flexibility/deflection of proximal opening 330a as tube 23 contacts bushing 330 and bends.

In FIG. 4E, bushing 330 includes proximal section 331 and sloped section 333 that extends between proximal section 331 and distal end 330b. Accordingly, bushing 330 in FIG. 4E may be generally similar to bushing 330 in FIGS. 3A and 3B but without curved section 332.

In FIG. 4F, bushing 330 includes a sloped section 333 that extends between distal end 330b and proximal opening 330a and is therefore similar to bushing 330 in FIGS. 4A and 4B. However, in FIG. 4F, sloped section 333 is configured to intersect the outer surface of bushing 330 at proximal opening 330a to thereby maximize the diameter of proximal opening 330a.

In FIG. 4G, bushing 330 includes proximal section 331 and sloped section 333 that extends between proximal section 331 and distal end 330b. However, unlike bushing 330 in FIG. 4E, the proximal end of proximal section 331 is curved. In other words, proximal section 331 has a concave cross-sectional shape which can facilitate advancing the tip of tube 23 into bushing 330 when the tip may initially contact the proximal face of bushing 330.

FIGS. 5A and 5B provide another example of a variation of bushing 330 that could be used in a ported catheter, or another PIVC, to facilitate insertion of a blood draw device through the ported catheter, or other PIVC, in accordance with embodiments of the present disclosure. Bushing 330 in FIGS. 5A and 5B includes a curved section 401 that extends between distal end 330b and proximal opening 330a. Curved section 401 can have a concave cross-sectional shape such that the inside diameter of bushing decreases along a curve from proximal opening 330a towards distal end 330b. The thickness of the walls of bushing 330 may be generally consistent and therefore the design of bushing 330 in FIGS. 5A and 5B may be suitable when bushing 330 is formed of metal.

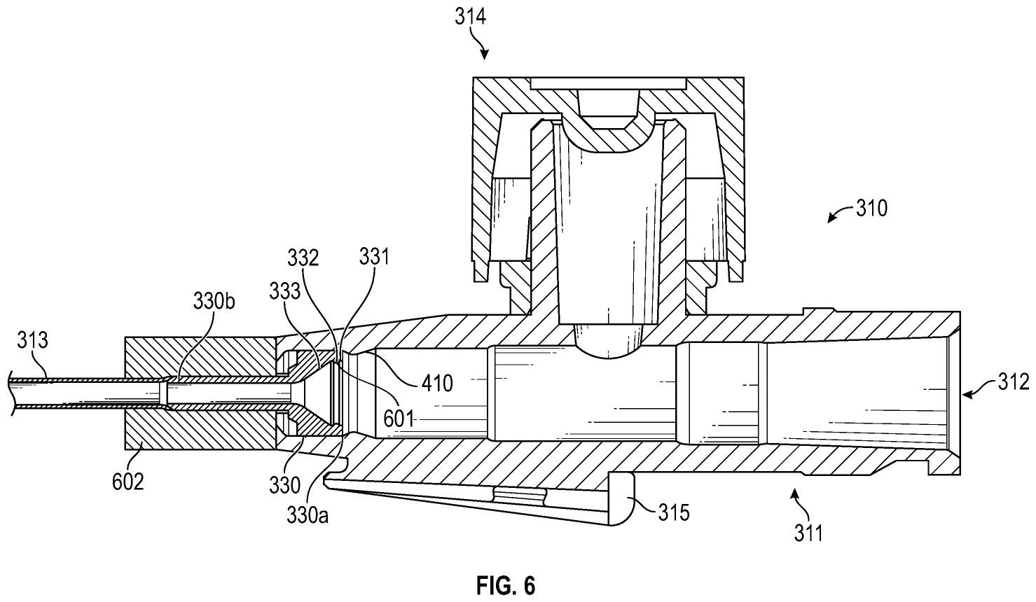

FIG. 6 is a cross-sectional view of a ported catheter 310 that includes a bushing 330 that is configured to facilitate insertion of a blood draw device through ported catheter 310 and that includes a catheter strain relief feature 602 in accordance with one or more embodiments of the present disclosure. In this embodiment, port 314 is oriented at a perpendicular angle relative to the longitudinal axis of catheter adapter 311.

Bushing 330 in FIG. 6 includes proximal section 331, curved section 332, and sloped section 333 that are configured similar to what is described above with reference to FIGS. 3A and 3B. Additionally, a rounded section 601 may protrude from proximal section 331 forming an annular ring of reduced diameter inside of proximal opening 330a and proximal to curved section 332. Rounded section 601 may facilitate advancing tube 23 into bushing 330 when blood draw device 20 is connected to port 314 at a perpendicular angle.

Ported catheter 310 in FIG. 6 also includes catheter strain relief feature 602 that extends around catheter 313 distal to catheter adapter 311. Catheter strain relief feature 602 may be a rubberized or otherwise flexible material that is molded or otherwise secured around catheter 313 to reinforce the portion of catheter 313 immediately distal to catheter adapter 311. By reinforcing this portion of catheter 313, tube 23 can be less likely to kink when it is extended through catheter 313.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A ported catheter comprising:
a catheter adapter having a proximal opening;
a catheter extending distally from the catheter adapter;
a port; and
a bushing having an inside surface that is configured to facilitate insertion of a tube of a blood draw device or other vascular probe or instrument into the catheter.

2. The ported catheter of claim 1, wherein the inside surface of the bushing includes a curved section.

3. The ported catheter of claim 2, wherein the curved section has a concave cross-sectional shape.

4. The ported catheter of claim 2, wherein the inside surface of the bushing also includes a proximal section positioned at a proximal opening of the bushing, the curved section extending distally from the proximal section.

5. The ported catheter of claim 4, wherein the inside surface of the bushing also includes a sloped section that extends proximally from the curved section.

6. The ported catheter of claim 5, wherein the port is angled towards the bushing.

7. The ported catheter of claim 1, wherein the inside surface of the bushing includes a sloped section that extends between a distal end of the bushing and a proximal opening of the bushing.

8. The ported catheter of claim 7, wherein the sloped section intersects with an outer surface of the bushing at the proximal opening.

9. The ported catheter of claim 1, wherein the inside surface of the bushing includes a curved section that extends between a distal end of the bushing and a proximal opening of the bushing, the curved section having a convex cross-sectional shape.

10. The ported catheter of claim 1, wherein the inside surface includes a proximal section positioned at a proximal opening of the bushing and a sloped section that extends between the proximal section and a distal end of the bushing.

11. The ported catheter of claim 10, wherein a proximal end of the proximal section is curved.

12. The ported catheter of claim 1, wherein the inside surface includes a proximal section positioned at a proximal opening of the bushing, a rounded section within the proximal section, a curved section that extends distally from the proximal section, and a sloped section that extends between the curved section and a distal end of the bushing.

13. The ported catheter of claim 1, wherein the bushing is formed of plastic, metal, or rubber.

14. The ported catheter of claim 1, further comprising:
a catheter strain relief feature formed around the catheter distal to the catheter adapter.

15. A bushing for a ported catheter comprising:
a distal end that is configured to secure a catheter within a catheter adapter of the ported catheter;
a proximal opening; and
an inside surface that is configured to facilitate insertion of a tube of a blood draw device or other vascular probe or instrument into the catheter.

16. The bushing of claim 15, wherein the inside surface includes a curved section.

17. The bushing of claim 16, wherein the inside surface also includes a sloped section and a proximal section, and wherein the curved section is positioned between the sloped section and the proximal section.

18. The bushing of claim 16, wherein the curved section extends between the proximal opening and the distal end.

19. The bushing of claim 16, wherein the inside surface includes a slanted section.

20. A system for collecting a blood sample comprising:
a ported catheter, and
a blood draw device having a tube that is configured to extend through the ported catheter,
wherein the ported catheter includes a bushing having an inside surface that is configured to facilitate inserting the tube through the ported catheter.
